(19) United States
(12) Patent Application Publication
(10) Pub. No.: US 2007/0275424 A1
(43) Pub. Date: Nov. 29, 2007

(54) Diagnostic Tests and Methods for Diagnosing Inflammatory Bowel Disease

(76) Inventors: Andrew Gewirtz, St Atlanta, GA (US); Shanti Sitaraman, St. Atlanta, GA (US); Jan Michael Klappseth, St. Atlanta, GA (US)

Correspondence Address: Christopher B Linder;Thomas Kayden Horstemeyer & Risley, 100 Galleria parkway N W, Suite 1750, Atlanta, GA, 30339-5948, US

(21) Appl. No.: 10/538,034

(22) PCT Filed: Nov. 24, 2003
(86) PCT No.: PCT/US03/37749

§ 371 Date: Apr. 12, 2007

Related U.S. Application Data
(60) Provisional application No. 60/428,521, filed on Nov. 22, 2002.

(51) Int. Cl. G01N 33/53

(52) U.S. Cl. 435/7.92; 436/503

(57) ABSTRACT

Methods are disclosed for diagnosing inflammatory bowel disease (IBD), one exemplar method of which includes determining whether a sample is positive for anti-flagellin antibodies (AFA), diagnosing the presence of IBD when the sample is positive for AFA, and diagnosing the absence of IBD when the sample is negative for AFA. Alternatively, instead of testing for positivity with respect to AFA, or in addition thereto, an exemplar method can determine whether the sample has an AFA level above an AFA cut-off value (X); and diagnosing IBD when the AFA level is above X, and diagnosing absence of IBD when the AFA level is below X. Test kits that include items used to carry out the disclosed methods are also disclosed.

### CLAIM OF PRIORITY

This application claims priority to copending U.S. provisional application entitled, “Diagnostic Test for Inflammatory Bowel Disease,” having Ser. No. 60/428,521, filed on Nov. 22, 2002, which is entirely incorporated herein by reference.

### TECHNICAL FIELD

This disclosure relates generally to the fields of inflammatory bowel disease and immunology and, more specifically, to methods for distinguishing inflammatory bowel disease from other disorders.

### BACKGROUND OF THE INVENTION

Inflammatory bowel disease (IBD), which occurs worldwide and afflicts millions of people, is the collective term used to describe two gastrointestinal disorders of unknown etiology: Crohn's disease (CD) and ulcerative colitis (UC). IBD together with irritable bowel syndrome (IBS) will affect one-half of all Americans during their lifetime, at a current approximate cost of greater than $2.6 billion dollars for IBD and greater than $8 billion dollars for IBS. A primary determinant of these high medical costs is the difficulty of diagnosing digestive diseases. The cost of IBD and IBS is compounded by lost productivity, with persons suffering from these disorders missing at least eight more days of work annually than the national average.

Inflammatory bowel disease has many symptoms in common with irritable bowel syndrome, including abdominal pain, chronic diarrhea, weight loss and cramping, making definitive diagnosis extremely difficult. Of the five million people suspected of suffering from IBD in the U.S., only about one million are diagnosed as such. The difficulty in differentially diagnosing IBD and IBS hampers early and effective treatment of these diseases. Thus, there is a need for rapid and sensitive testing methods for definitively distinguishing IBD from IBS.

Progress has been made in precisely diagnosing, in many cases, Crohn's disease and ulcerative colitis. Current methods, however, for diagnosing an individual as having Crohn's disease or ulcerative colitis, while highly specific, are relatively costly, requiring labor-intensive immunofluorescence assays and careful analysis of cell staining patterns. Further, IBD is currently primarily diagnosed via colonoscopy, which is an expensive and complex procedure requiring anesthesia In addition to causing discomfort to the patient, it can typically be a lengthy amount of time before results can be obtained. Often, it takes months from the time the physician orders the test until the results are obtained and presented to the patient.

Although these costly assays are easily justified for those individuals previously diagnosed with or strongly suggested to have IBD, a less expensive but highly sensitive alternative would be advantageous for first determining if an individual has inflammatory bowel disease at all.

Unfortunately, such a highly sensitive and inexpensive primary screening test for distinguishing IBD from other digestive diseases presenting with similar symptoms is not used ubiquitously. There are currently at least two serological IBD diagnostic tests being commercially marketed by Prometheus Labs, Inc. (San Diego, Calif., USA). At least one of these tests is focused on determining the presence or levels of antibodies to human proteins and to yeast, which involves multiple testing of the human. However, while such currently commercially used tests are useful, their usefulness is limited by their modest sensitivity. Thus, tests with greater sensitivity and/or which can compliment the sensitivity of existing tests are needed.

Crohn's disease and ulcerative colitis (collectively, IBD), are characterized by seemingly aberrant mucosal immune responses. Namely, while the active flares of disease activity resemble both clinically and histopathologically the acute food-borne gastroenteritis caused by enteric pathogens, efforts to associate specific pathogens with IBD have been unsuccessful.

Thus, there is a need for better methods of rapidly and inexpensively diagnosing inflammatory bowel disease and distinguishing between subtypes therein. A heretofore unaddressed need exists in the industry to address the aforementioned deficiencies and inadequacies.

### SUMMARY

The present disclosure provides test kits and methods for diagnostic testing for inflammatory bowel disease (IBD). In this regard, one embodiment of such a method, among others, can be broadly summarized by the following: providing a sample from an individual; determining whether the sample is positive for anti-flagellin antibodies (AFA); and diagnosing whether the individual as likely having IBD when the sample is positive for AFA, and diagnosing the individual as probably not having IBD when the sample is negative for AFA. This test has stand-alone diagnostic value and may compliment the diagnosis of existing serologic and other tests. Alternatively, instead of testing for positivity with respect to AFA, the method may determine whether the sample has an AFA level above an AFA cut-off value (X); and diagnosing the individual as having IBD when the AFA level is above X, and diagnosing said individual as not having IBD when the AFA level is below X. Antibodies exist in various forms such as IgG or IgA. Thus, both IgG and IgA AFA can be measured, which can increase the accuracy of the test. Preferably the sample is a serological sample.

Briefly described, one embodiment of the test kit, among others, can include the following items: a substrate comprising a coating of purified flagellin; a standard with a known concentration of AFA; a detection antiserum labeled with a chromogenic indicator capable of color development when exposed to a developing solution; a developing solution; an assay wash buffer; and a color chart indicating AFA concentrations corresponding to a plurality of discernible colors.

Other kits, methods, features, and advantages of this disclosure will be or become apparent to one with skill in the art upon examination of the following drawings and detailed description. It is intended that all such additional kits, methods, features, and advantages be included within this description, be within the scope of this disclosure, and be protected by the accompanying claims.

### BRIEF DESCRIPTION OF THE DRAWINGS

This disclosure can be better understood with reference to the following drawings. The components in the drawings are not necessarily to scale, emphasis instead being placed upon clearly illustrating the principles of this disclosure. Moreover, in the drawings, like reference numerals designate corresponding parts throughout the several views.

FIG. 1 is a graph of serum antibody titers measured via ELISA to E. coli flagellin and E. coli lipopolysaccharide (LPS) in a control individual and in an individual with inflammatory bowel disease (IBD).

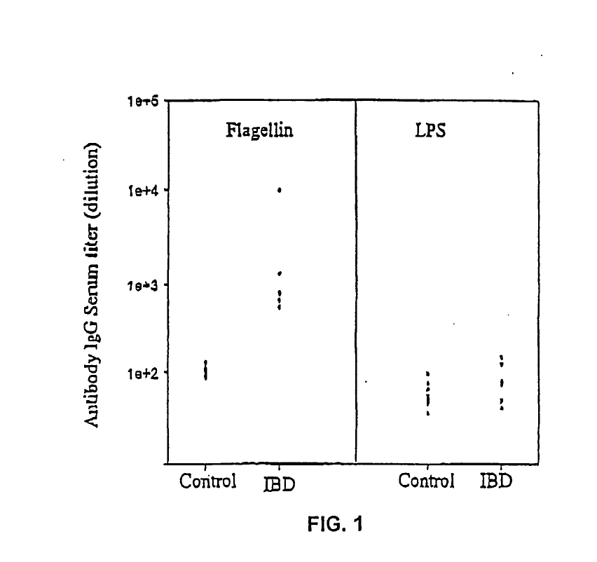

FIG. 2 (A&B) are SDS-PAGE immunoblots of lysates of flagellated or unflagellated whole bacteria (A) or purified flagellin, using serum from control or IBD subjects as primary antibody.

FIG. 3 illustrates plots of results of flagellin-specific, LPS-specific, and total Ig levels assayed.

### DETAILED DESCRIPTION

A highly sensitive primary screening test would provide physicians with an inexpensive methods for rapidly distinguishing individuals with inflammatory bowel disease (IBD) from those having irritable bowel syndrome (IBS) and would facilitate earlier and more appropriate therapeutic intervention and minimize uncertainty for patients and their families. If desired, such a primary screening assay could be combined with a subsequent, highly specific assay for determining if an individual diagnosed with IBD has Crohn's disease or ulcerative colitis. Disclosed herein are sensitive new methods and kits for serologically testing for the presence of IBD.

IBD patients have been shown to have an elevated level of immunoreactivity to their own microflora and in multiple murine models of IBD, intestinal inflammation does not develop if mice were used in the models are maintained in germ-free conditions. Thus, IBD can be mediated by aberrant immune responses directed at presumably normal enteric microflora. The specific microbial targets to which this elevated mucosal immune response are directed aid in diagnosing, understanding, and perhaps treating IBD.

For several reasons, bacterial flagellin is involved in the mucosal immune responses associated with IBD. Being expressed abundantly by all motile bacteria, flagellin is present in substantial amounts in the intestine. Flagellin is highly antigenic being a major immunoglobulin target in a variety of infectious events. Flagellin is a potent and direct activator of the innate immune system. Specifically, recognition of flagellin by the germ-line encoded pattern recognition receptor toll-like receptor 5 (TLR5), which is expressed on the basolateral membrane of polarized intestinal epithelial cells, can directly promote a mucosal inflammatory response. Flagellin's ability to activate the innate immune system likely underlies its antigenicity and may also allow it to act as an adjuvant promoting CD4 T-cell responses to bystander molecules.

### Definitions

Some terms used herein have specific definitions for the purposes of this document.

As used herein, the term “antibody” means a population of immunoglobulin molecules, which can be polyclonal or monoclonal and of any isotype. As used herein, the term “antibody” encompasses an immunologically-active fragment of an immunoglobulin molecule. Such an immunologically-active fragment contains the heavy and light chain variable regions, which make up the portion of the antibody molecule that specifically binds an antigen. For example, an immunologically-active fragment of an immunoglobulin molecule known in the art as Fab, Fab′, or F(ab′)₂ is included within the meaning of the term “antibody.”

As used herein, the term “inflammatory bowel disease” is synonymous with “IBD” and is a collective term referring to both Crohn's disease and ulcerative colitis. Thus, an individual having either Crohn's disease or ulcerative colitis is defined herein as having IBD. Conversely, an individual having neither ulcerative colitis nor Crohn's disease does not have IBD as defined herein. The term “inflammatory bowel disease” distinguishes Crohn's disease and ulcerative colitis from all other disorders, syndromes, or abnormalities of the gastroenterological tract, including irritable bowel syndrome (IBS).

As used here, in the term “anti-flagellin antibodies” is synonymous with “AFA” and means antibodies to any flagellin. The flagellin may be, for example, E. coli flagellin. AFA, such as serum or saliva AFA, can be detected using an enzyme-linked immunosorbent assay (ELISA) with fixed flagellin, for example (see Example I). The term AFA as used herein encompasses all varieties of anti-flagellin reactivity. Similarly, the term AFA encompasses all immunoglobulin isotypes including, for example, immunoglobulin A and G.

In the methods of this disclosure, a sample to be analyzed is obtained from the individual to be diagnosed. The term “sample,” as used herein, means any biological specimen obtained from an individual that contains antibodies. A sample can be, for example, whole blood, plasma, saliva or other bodily fluid or tissue having antibodies, preferably a serum sample. The use of a serum sample is described in Example I; the use of other samples, such as saliva and urine samples, is well known in the art (see, for example, Hashida et al., J. Clin. Lab. Anal. 11:267-86 (1997), which is incorporated by reference herein). One of ordinary skill in the art understands that samples such as serum samples can be diluted prior to analysis of AFA content.

As used herein, the term “histological analysis of flagellin” means any technique revealing the structure of a flagellinic cell using staining or microscopy. Histological analysis, which encompasses techniques such as immunocytochemistry and indirect immunofluorescence, as well as other methods involving microscopy, is explicitly excluded from the scope of the present disclosure. In contrast, ELISA, in which flagellin reactivity is analyzed by means of a detectable secondary antibody that generates a quantitative signal, does not involve microscopy or other analysis of cell structure and therefore is not “histological analysis of flagellin” as defined herein.

### Discussion

In investigating the pathogenesis of inflammatory bowel disease (IBD), it has been observed that individuals with IBD have substantially elevated serum antibody titers to bacterial protein flagellin (hereinafter, flagellin), e.g., E. coli flagellin. Disclosed herein are methods for using enzyme-linked immunosorbent assays (ELISA) to diagnose IBD. The methods do not include labor-intensive immunofluorescence analysis of fixed flagellin or other flagellin histological analysis. Thus, the disclosed methods provide a rapid and sensitive methods of differentiating individuals having either Crohn's disease (CD) or ulcerative colitis (UC) from those who do not have IBD. The disclosed methods can be used alone to rule out IBD in an individual suspected of having the disease, or, when positive for diagnosis of IBD, can be used in combination with a subsequent assay disclosed herein that specifically differentiates CD from UC.

Thus, disclosed are highly sensitive methods of diagnosing IBD in an individual. One exemplar method includes the steps of providing a sample from the individual; determining by non-histological means whether the sample is positive for anti-flagellin antibodies (AFA) or determining the concentration level of AFA present; and diagnosing the individual as having IBD when the sample is positive for AFA, or a certain concentration level of AFA is reached. Alternatively, one disclosed method step can be diagnosing the individual as not having IBD when the sample is negative for AFA or if the AFA concentration level is below a certain amount, provided that the method does not include histological analysis of flagellin. In the disclosed methods, AFA positivity or concentration level can be determined using an immunoassay, for example.

If IBD is diagnosed via the concentration level of AFA, then the level of AFA in the patient sample is usually compared with at least one standard. Preferably, the concentration level of AFA in the blood/serum sample is compared with a set of standards or via a laboratory spectrophotometer such as an EMAX Microplate Reader™ (Molecular Devices, Menlo Park, Calif.). Thus, disclosed are highly sensitive methods of diagnosing inflammatory bowel disease, which methods do not necessarily include histological analysis of flagellin.

A variety of assay formats can be used to determine AFA levels in a patient sample. The disclosed methods can be performed with whole cells, such as flagellin cells, for the determination of AFA levels; with unpurified or partially purified cell extracts; or with purified proteins, protein fragments or peptides, which can be produced, for example, recombinantly, synthetically or using phage display technology.

Flow cytometry can be used to determine AFA levels according to an exemplary disclosed method. Such flow cytometric assays, including bead-based immunoassays, can be used to determine AFA levels in the same manner as used to detect serum antibodies to Candida albicans and serum antibodies to HIV proteins (see, for example, Bishop and Davis, J. Immunol. Methods 210:79-87 (1997); McHugh et al., J. Immunol. Methods 116:213 (1989); Scillian et al., Blood 73:2041 (1989), each of which is incorporated by reference herein).

Phage display technology for expressing a recombinant antigen specific for AFA also can be used to determine the level of AFA. Phage particles expressing the antigen specific for AFA can be anchored, if desired, to a multiwell plate using an antibody such as an antiphage monoclonal antibody (Felici et al., “Phage-Displayed Peptides as Tools for Characterization of Human Sera” in Abelson (Ed.), Methods in Enzymol. 267, San Diego: Academic Press, Inc. (1996), which is incorporated by reference herein).

A variety of immunoassay formats including competitive and non-competitive immunoassay formats also are useful in the disclosed methods (Self and Cook, Curr. Opin. Biotechnol. 7:60-65 (1996), which is incorporated by reference). Immunoassays encompass capillary electrophoresis based immunoassays (CEIA) and can be automated, if desired. Immunoassays also can be used in conjunction with laser-induced fluorescence (see, for example, Schmalzing and Nashabeh, Electrophoresis 18:2184-93 (1997)); Bao, J. Chromatogr. B. Biomed. Sci. 699:463-80 (1997), each of which is incorporated herein by reference). Liposome immunoassays, such as flow-injection liposome immunoassays and liposome immunosensors, also can be used to determine AFA levels according to exemplar methods (Rongen et al., J. Immunol. Methods 204:105-133 (1997), which is incorporated by reference herein).

Immunoassays, such as ELISAs, can be used in one exemplar method. A fixed flagellin ELISA, for example, can be useful for determining whether a sample is positive for AFA or for determining the AFA level in a sample (see Example I). An enzyme such as for example, but not limited to, horseradish peroxidase (HRP), alkaline phosphatase (AP), β-galactosidase or urease can be linked to a secondary antibody selective for AFA, or for the invariant portion of a human antibody, for use in an exemplar method. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product that is readily detectable at 405 nm. Similarly, a β-galactosidase detection system can be used with the chromogenic substrate o-nitrophenyl-β-D-galactopyranoside (ONPG), which yields a soluble product that is detectable at 410 nm, or a urease detection system can be used with a substrate such as urea-bromocresol purple (Sigma Immunochemicals, St. Louis, Mo., USA). A useful secondary antibody linked to an enzyme can be obtained from a number of commercial sources; goat F(ab′)₂ anti-human IgG-alkaline phosphatase, for example, is commercially from Jackson Immuno-Research (West Grove, Pa., USA), or a rabbit anti-human serum may be employed as well.

A radioimmunoassay also can be useful for determining whether a sample is positive for AFA, or for determining the level of AFA in a sample. A radioimmunoassay using, for example, an iodine-125 labeled secondary antibody (Harlow and Lane, Antibodies A Laboratory Manual Cold Spring Harbor Laboratory: New York, 1988, which is incorporated herein by reference) is encompassed within this disclosure.

A secondary antibody labeled with a chemiluminescent marker also can be useful in the disclosed methods. Such a chemiluminescent secondary antibody is convenient for sensitive, non-radioactive detection of AFA and can be obtained commercially from various sources such as Amersham Biosciences, Inc. (Piscataway, N.J., USA).

In addition, a detectable reagent labeled with a fluorochrome can be useful in the disclosed methods for determining whether AFA is present in a sample. Appropriate fluorochromes include, for example, DAPI, fluorescein, Hoechst 33258, R-phycocyanin, B-phycoerythrin, R-phycoerythrin, rhodamine, Texas red or lissamine. A particularly useful fluorochrome is fluorescein or rhodamine. Secondary antibodies linked to fluorochromes can be obtained commercially. For example, goat F(ab′)₂ anti-human IgG-FITC is available from Tago Immunologicals (Burlingame, Calif., USA).

A signal from the detectable reagent can be analyzed, for example, using a spectrophotometer to detect color from a chromogenic substrate; a radiation counter to detect radiation, such as a gamma counter for detection of iodine-125; or a fluorometer to detect fluorescence in the presence of light of a certain wavelength. For detection of enzyme-linked reagents, a quantitative analysis of the amount of AFA can be made using a spectrophotometer such as an EMAX Microplate Reader (Molecular Devices, Menlo Park, Calif.) in accordance with the manufacturer's instructions. If desired, the assays of this disclosure can be automated or performed robotically, and the signal from multiple samples can be detected simultaneously. Immunoassays using a secondary antibody selective for AFA can be used in the disclosed methods.

It will be understood to one of ordinary skill in the art that the disclosed methods can be modified to determine particular types of IBD, such as CD or UC, if it is determined that each of these diseases is characterized by a particular level of AFA concentration. Thus, by determining the exact concentration level of AFA present, an exemplar method is able to diagnose which of the types of IBD the individual has that is being tested.

Test kits are also included in the disclosed embodiments. An exemplary test kit includes a substrate comprising a coating of purified flagellin; a standard with a known concentration of AFA; a detection antiserum labeled with a chromogenic indicator capable of color development when exposed to a developing solution; a developing solution; and/or an assay wash buffer. In addition, the test kit can include instructions for use. Optionally, the test kit and/or instructions may further include a color chart indicating AFA concentrations corresponding to a plurality of discernible colors.

The following example is intended to illustrate but not limit the disclosed methods and tests.

### EXAMPLE I

### Determination of Patient AFA Status

Healthy humans mount a highly effective innate immune inflammatory response to a variety of enteric pathogens that, although causing substantial temporary distress to the host, generally clears the infection from the intestinal mucosa without substantial systemic colonization. A similar immune inflammatory response occurs in repeated sporadic flares in inflammatory bowel disease in the absence of any known pathogen perhaps being triggered by commensal intestinal microbes. The intestinal epithelium actively promotes this immune inflammatory response, particularly via secretion of pro-inflammatory chemokines.

Studies into the mechanisms that regulate intestinal epithelial chemokine secretion have shown that the pathogen Salmonella typhimurium, but not commensal gut microbes activate the pro-inflammatory transcription factor NF-κB thus resulting in the chemokine secretion. This NF-κB activation results primarily from epithelial contact with bacterial flagellin, a structural component of flagella. Such flagellin is secreted by most gram-negative bacteria, including for example, commensal gut E. coli, and thus flagellin is ubiquitous at the apical (lumenal) membrane domain of the intestinal epithelium. Flagellin, however, can only activate epithelial chemokine secretion when it reaches the basolateral membrane and such flagellin translocation across model epithelia can be mediated by the pathogen S. typhimurium but not by commensal E. coli.

Flagellins amongst many bacterial species are well conserved, and thus a common serotype of E. coli flagellin was selected to serve as a “generic,” or exemplary, flagellin. Although samples from all persons assayed using the disclosed methods exhibited detectable immunoreactivity to this common flagellin, the level of the response was substantially elevated in patients with Crohn's disease.

It was found that the epithelial response to flagellin is mediated by the pattern recognition receptor toll-like receptor 5 (TLR5), a receptor whose expression is highly polarized to the basolateral surface. In addition to promoting acute inflammation, flagellin can also promote adaptive immune responses, in particular serving as an adjuvant for CD4 T cells. Flagellin promotes such T cell responses via acting on dendritic cells, which also express TLR5. Evidence of the potential role of flagellin in IBD can be seen by the elevated titer of AFA in this disorder. Thus, flagellin likely plays a role in the acute inflammation that occurs in both Salmonellosis and IBD. Further, flagellin likely promotes development of the autoreactive CD4 T cells that are believed to mediate the chronic phases of IBD.

Thus, this example describes analysis of patient AFA levels using an ELISA assay. While the present example analyzes AFA presence/levels using ELISA, other methods of detecting AFA presence/levels can also be used. For example, flow cytometry, phage display technology; immunoassays, including competitive and non-competitive immunoassays such as CEIA and liposome immunoassays; laser-induced fluorescence; radioimmunoassays; chemiluminescent markers; and fluorochromatic assays can be used to detect AFA presence/levels.

In the present exemplar method, an ELISA plate is provided that is coated with flagellin. A sample is collected from an individual. The sample can be serum. The serum is applied to the ELISA plate. AFA antibodies present in the sample will bind to the flagellin. The plate is washed with an assay wash buffer. Then a serum, preferably a rabbit serum that has been engineered to bind to the invariant region of human antibodies is applied to the plate. The rabbit serum includes therein a detecting molecule, e.g., HRP, which will change color in the presence of a developing solution (this solution is known as HRP-conjugated rabbit anti-human antiserum). The amount of HRP-conjugated rabbit anti-human antiserum that adheres to the plate will be proportional to the amount of AFA in the sample. Optionally, the plate is then washed with an assay wash buffer. The developing solution is contacted to the plate, and a determination is made whether a color change occurs. If a color change occurs, this indicates that AFA is present in the human sample. Depending on the color change, as can be measured visually, or quantitatively via a spectrophotometer, the level of AFA concentration can be determined as well, if the color is compared to the color of a standard with a known concentration of AFA.

### EXAMPLE II

### Specific Quantitive Analysis of AFA in IBD Patients

A. Methods

Reagents: Flagellin was chromatographically purified from S. typhimurium SL3201 or E. coli F-18 and its purity verified. E. coli LPS was purchased from Sigma-Aldrich Corp. (St. Louis, Mo.). Flagellin antibody was affinity purified from sera of rabbits injected with 3 monthly 100 microgram injections of E. coli flagellin.

Patients and sera: Immunoblotting was performed on IBD patients and healthy control volunteers. ELISAs were performed on 177 serum samples from the collection compiled by the Cedars-Sinai Medical Center Inflammatory Bowel Disease Center. Diagnosis of these patients and characterization of various 1BD serologic markers has been previously described in Landers et al, Gastroenterology 123:689-99 (2002). Control subjects are healthy individuals cohabitating with IBD patients. Samples remained coded until assays described below were complete.

SDS-PAGE Immunoblotting: Whole bacterial lysates (10⁷ CFU/ml) or purified flagellin (500 ng/ml) were analyzed by SDS-PAGE immunoblotting (10 μl per lane) using human serum from control or IBD subjects diluted 1:500 (unless indicated otherwise) as the primary antibody followed by anti-human IgG (Amersham, 1:5000) and ECL, detection (Amersham). Flagellate and aflagellate Salmonella were, respectively, S. typhimurhim strain SL3201 and isogenic fliC/fljB mutant. Flagellate and aflagellate E. coli were E. coli F-18 and E. coli-Top 10 (Invitrogen).

ELISA: Microtiter plates (from ICN, Carlsbad, Calif.) were coated with E. coli flagellin (100 ng/well), E. coli LPS 2 μg/well, or protein L (1 μg/well) applied overnight in 0.1 M NaHCO₃. Following overnight coating (performed on many plates), all plates were frozen at −20° C. and thawed daily as utilized. Sera from 177 subjects were diluted in ELISA wash buffer (HBSS with 0.5% goat serum, 0.1% Tween-20) 1:100, 1:500, and 1:10⁵. The lower 2 dilutions were applied to wells coated with LPS and flagellin while serum diluted 1:10⁵ was applied to wells coated with protein L. Each plate utilized a control serum sample that was prepared in bulk, aliquoted, frozen, and thawed daily as utilized. Following 1 h incubation, total or product-specific Ig was detected using anti-human IgG-HRP (1:5000) (Amersham) or two-step method of anti-human IgA-biotin (1:5000, Jackson Immuno) followed by avidin-peroxidase (1:10,000, Jackson Immuno). Peroxidase was then revealed via TMB substrate (KPL) and OD read at 650 nm. Data in figures is OD for LPS-specific and total Ig (at the 1:100 and 1:10⁵ dilutions, respectively) corrected only by normalizing to the plates control sample's values relative to the average control values over all the plates. For flagellin-specific Ig, the values are similarly reported except that when data values for the 1:100 dilution exceeded 0.6 (linear limit based on pilot analysis), values of 1:500 (multiplied by 5) were utilized. P-values were determined by Student's T-test.

B. Results and Discussion

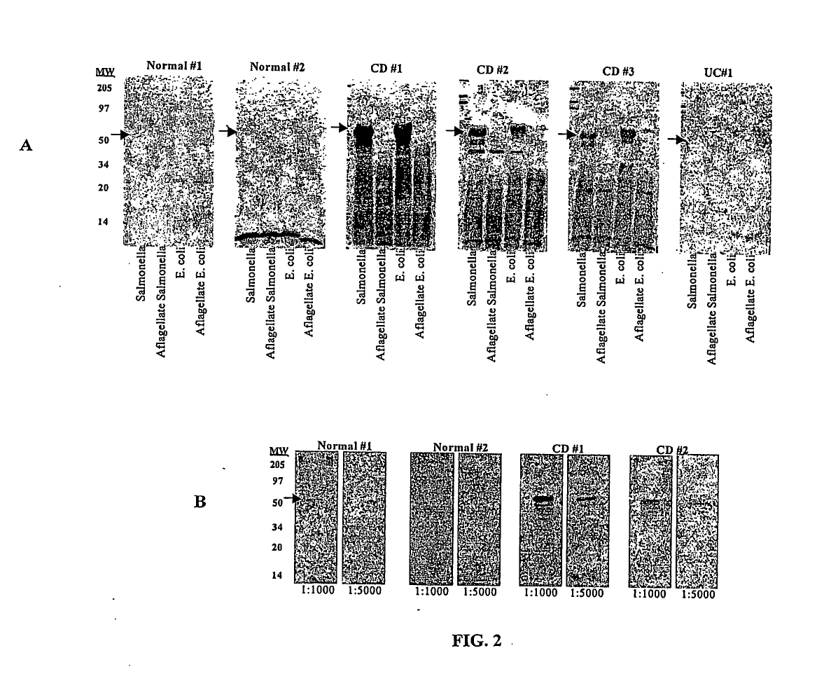

Flagellin is a target of the adaptive immune response in IBD. This principle was examined by using serum from IBD patients and normal controls to immunoblot bacterial extracts of flagellated and non-flagellated enteric bacteria as well as purified flagellin. As shown in FIG. 2, patients with CD exhibited a substantial general increase in immunoreactivity to these bacterial extracts. The arrows indicate the location of flagellin. The data are representative blots done with 6 control and 7 IBD subjects.

While increase in immunoreactivity was clearly observed for many components of these bacteria, the band appearing to be flagellin (based on its molecular weight) clearly appeared to be one of the specific targets. The band was verified to be flagellin by observing its absence in corresponding aflagellate bacteria and by use of highly purified polyclonal and monoclonal flagellin-specific antiserum (data not shown). Consistent with flagellin being one of the targets of the CD-associated immune response, immunoblotting of flagellin chromatographically purified from E. coli (FIG. 2B) or Salmonella (not shown) with these serum samples indicated CD patients had increased immunoreactivity to flagellin, consistent with flagellin being a highly conserved protein in general, and among these gram-negative bacteria in particular.

Flagellin appeared to be a target of the enhanced immune response associated with CD and flagellin's potential to drive immune responses to bystander antigens. A more quantitative and larger-scale analysis of flagellin immunoreactivity in IBD patients and control subjects was performed. Specifically, the levels of flagellin-specific immunoglobulin (IgG and IgA) in control and IBD patients were measured by ELISA. Total levels of IgG and IgA were also measured, as were levels of IgG and IgA that recognized LPS, as some studies have observed these to be elevated in IBD.

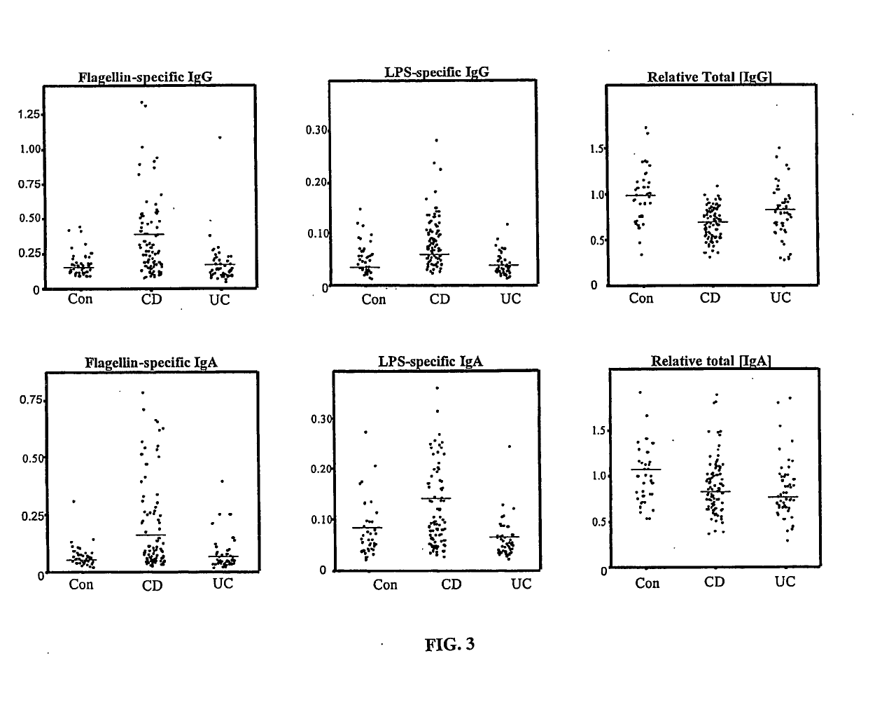

The specificity of these interactions was verified by observing that no immunoreactivity was observed to microtiter plates coated with BSA (not shown). While nearly all of the serum samples analyzed exhibited an easily quantifiable level of Ig that recognized flagellin, the levels of flagellin-specific Ig were significantly elevated in patients with CD (FIG. 3). The data shown in FIG. 3 are from 177 samples assayed in this Example. Means are represented by lines. Specifically, mean relative values of anti-flagellin IgG and IgA were increased by 2.5-fold and 3-fold, respectively, (p<10⁻⁵) with 52% of patients with CD exhibiting levels of flagellin-specific IgA or IgG that were higher than 2 standard deviations beyond the mean of the range exhibited by control persons who share a similar environment (levels of 2/40 control patients also met this criteria, i.e., 95% specificity). This increase was in spite of a total 30% decrease in mean total IgG (p<10⁻⁹); mean total IgA decreased by 25%, but the difference had only moderate statistical significance (p=0.02).

Consistent with previous studies, an increase in LPS-specific Ig was observed, although the relative mean increase (1.6- and 1.7-fold for IgA and IgG, respectively, p<002) and % of individuals exhibiting significantly elevated responses (26% of CD subjects had LPS-specific IgG or IgA>2 SD above mean of control subjects) was less than that observed for flagellin. Furthermore, the relative overall level of LPS-specific Ig was about 5-fold less than that observed for flagellin, despite using 20-fold more LPS to coat the microtiter plates (necessary to get measurable responses). Thus, the CD-associated immune response, and adaptive immune response in general, appears to target flagellin more than LPS.

The patients with elevated flagellin-specific Ig (45/87 as defined above and referred to below as flag+) tended to correlate positively with immunoreactivity for defined CD-specific and bacterial-derived antigens. Specifically, all 30 ASCA+CD patients tested were flag+, while only 15 of 57 ASCA−CD patients were flag+. Patients who were positive for the bacterial antigens Pseudomonas fluourescens protein 12 and OmpC also had high rates of flag+, 60% and 70% respectively. In contrast, of the 14 CD patients tested who were positive for the auto-antigen pANCA, only 1 was flag+. Thus, elevated immune responses to flagellin can be associated with CD markers and elevated immune responses to bacterial antigens in general, but not the autoantigen pANCA. Consistent with the principle that the specific bacterial antigens recognized in CD will vary among patients, we observed that of the 15 CD patients who had tested negative for all of the previously defined serologic markers, 3 tested flag+.

While innate immune activity, especially PMN infiltration, mediates the acute flares of IBD, chronic intestinal inflammation is characterized by increased numbers of mucosal adaptive immune cells, especially CD4 T-cells. While these T-cells are known to be broadly reactive to the intestinal microflora in general, the specific antigens that drive these T-cells are relatively unknown. For most antigens, generation of antibodies requires activating antigen-specific T-cells. Consistent with this, generation of flagellin-specific Ig in mice is absolutely T-cell dependent (TCR KO mice exhibit no flagellin-specific Ig following IP injection of high doses of flagellin—data not shown). Thus, due to CD patients exhibiting elevated levels of flagellin-specific Ig, flagellin can be considered an antigenic target of the elevated adaptive immune response that characterizes the chronic stage of inflammation in IBD. Flagellin can be especially important for the CD-associated immune response, in that it is one of the major targets of the immune response to enteric bacteria in general, as evidenced by our immunoblotting of whole extracts of flagellate and aflagellate bacteria with CD serum. Furthermore, flagellin has substantial adjuvant ability, and thus can be driving the immune responses to other bacterial antigens in the gut.

Adaptive immunity is thought to be absolutely dependent upon innate immunity. Thus, a molecule speculated to drive innate immunity in IBD that is also a target of adaptive immunity is consistent with this now nearly universally accepted immunologic principle. The innate immune response to flagellin is mediated by TLR5, which, without wishing to be bound to any theory, is thought to be present on the basolateral surface of the epithelium as well as on human dendritic cells (DC). While DC activation is presumably critical for activation of mucosal T-cells, this could occur in response to flagellin via activation of TLR5 on DC or via activation of epithelial TLR5 resulting in secretion of DC-activating cytokines. In support of a role for the latter, flagellin is an effective adjuvant in mice, even though mouse DC do not express significant levels of TLR5.

It should be emphasized that the above-described embodiments, particularly, any “preferred” embodiments, are merely possible examples of implementations, and are merely set forth for a clear understanding of the principles set forth herein. Additionally, the step of any disclosed method can, optionally, be performed out of the order disclosed herein, and the methods may omit certain steps altogether. Many variations, substitutions, and modifications may be made to the above-described embodiment(s) without departing substantially from the spirit and principles herein. All such variations, substitutions, and modifications are intended to be included herein within the scope of this disclosure and the following claims.

1. A method for diagnosing inflammatory bowel disease (IBD), comprising the steps of:
determining whether a sample obtained from a mammal is positive for anti-flagellin antibodies (AFA); and
diagnosing the individual as having IBD when the sample is positive for AFA, and diagnosing the individual as not having IBD when the sample is negative for AFA.

2. The method of claim 1, wherein the sample is chosen from blood, serum, saliva, stool and tissue.

3. The method of claim 1, wherein AFA positivity is determined using an enzyme-linked immunosorbent assay (ELISA).

4. The method of claim 1, wherein AFA positivity is determined using an assay type chosen from flow cytometry, phage display technology, immunoassays, laser-induced fluorescence, radioimmunoassays, chemiluminescent markers, and fluorochromatic assays.

5. The method of claim 4, wherein the immunoassay is chosen from a competitive immunoassay and a non-competitive immunoassay.

6. The method of claim 5, wherein the immunoassay is chosen from a capillary electrophoresis based immunoassay and a liposome immunoassay.

7. A method, comprising the steps of:
determining whether a sample obtained from a mammal has an anti-flagellin antibodies (AFA) level above an AFA cut-off value (X); and
diagnosing the individual as having inflammatory bowel disease (IBD) when the AFA level is above X, and diagnosing said individual as not having IBD when the AFA level is below X, wherein X is independently selected to achieve an optimized clinical parameter chosen from at least one of the following: sensitivity, specificity, negative predictive value, positive predictive value, and overall agreement.

8. The method of claim 7, wherein the sample is chosen from blood, serum, saliva, stool and tissue.

9. The method of claim 7, wherein AFA level is determined using an enzyme-linked immunosorbent assay (ELISA).

10. The method of claim 7, wherein AFA positivity is determined using an assay type chosen from flow cytometry, phage display technology, immunoassays, laser-induced fluorescence, radioimmunoassays, chemiluminescent markers, and fluorochromatic assays.

11. A method, comprising the steps of:
contacting an appropriate dilution of a sample obtained from a mammal with an antigen specific for anti-flagellin antibodies (AFA) under conditions suitable to form a complex of AFA and the antigen specific for AFA;
determining the amount of the complex; and
diagnosing the mammal as having IBD when the amount of the complex formed is greater than an AFA cut-off value (X), and diagnosing the mammal as not having IBD when the amount of the complex formed is less than X, wherein X is independently selected to achieve an optimized clinical parameter chosen from at least one of the following: sensitivity, specificity, negative predictive value, positive predictive value and overall agreement,
provided that said method does not include histological analysis of flagellin.

12. The method of claim 11, wherein the sample is at least one of a serum sample, a saliva sample, a blood sample, a stool sample, and a tissue sample.

13. The method of claim 11, wherein the antigen specific for AFA is fixed flagellin.

14. The method of claim 11, wherein the amount of the complex is determined using at least one of the following assays: an enzyme-linked immunosorbent assay (ELISA), flow cytometry, phage display technology, an immunoassays, laser-induced fluorescence, radioimmunoassays, chemiluminescent markers, and fluorochromatic assays.

15. A test kit for determining the concentration of anti-flagellin antibodies (AFA) present in a sample, comprising:
a substrate comprising a coating of purified flagellin;
a standard with a known concentration of AFA;
a detection antiserum labeled with a chromogenic indicator capable of color development when exposed to a developing solution;
a developing solution; and
an assay wash buffer.

16. The test kit of claim 15, further comprising a color chart indicating known AFA concentrations corresponding to a plurality of discernible colors.

17. The test kit of claim 15, further comprising directions for use.

18. A method, comprising the steps of:
contacting a sample obtained from a mammal with a substrate coated at least in part with purified flagellin;
contacting the substrate with a detection antiserum labeled with a chromogenic indicator capable of color development when exposed to a developing solution;
contacting the developing solution with the substrate; and
detecting the presence of a plurality of AFA.

19. The method of claim 18, further comprising the step of determining whether the mammal has IBD based on the presence or absence of AFA.

20. The method of claim 18, further comprising the step of determining whether the mammal has IBD based on an amount of AFA present.

21. A method for diagnosing inflammatory bowel disease (IBD), comprising the steps of:
determining whether a sample obtained from a mammal is positive for anti-flagellin antibodies (AFA); and
diagnosing the individual as likely having IBD when the sample is positive for AFA, and diagnosing the individual as probably not having IBD when the sample is negative for AFA.

22. The method of claim 21, wherein determining whether a sample obtained from a mammal is positive for AFA comprises measuring both IgG AFA and IgA AFA.
